(19) United States
(12) Patent Application Publication
(10) Pub. No.: US 2013/0108619 A1
(43) Pub. Date: May 2, 2013

(54) INTRAVENOUS IMMUNOGLOBULIN PROCESSING, DIAGNOSTIC, AND TREATMENT SYSTEMS AND METHODS

(71) Applicant: Isaac Melamed, Centennial, CO (US)

(72) Inventor: Isaac Melamed, Centennial, CO (US)

(21) Appl. No.: 13/668,010

(22) Filed: Nov. 2, 2012

Related U.S. Application Data
(60) Provisional application No. 61/554,760, filed on Nov. 2, 2011.

(51) Int. Cl. A61K 39/395; A61P 37/04; A61P 37/06; A61P 25/00; A61P 35/00; A61P 37/02; A61P 19/02

(52) U.S. Cl. 424/130.1

(57) ABSTRACT

A method of providing an immunotherapy treatment to a patient includes determining a level of C1 esterase inhibitor or inhibitor activity in the patient, determining an intravenous immunoglobulin dosing protocol for the patient based on the level of C1 esterase inhibitor or inhibitor activity, and administering the immunotherapy treatment to the patient.

### CROSS-REFERENCE TO RELATED APPLICATIONS

This application claims the benefit of U.S. Provisional Application No. 61/554,760 filed on Nov. 2, 2011, which is hereby incorporated in its entirety and for all purposes.

### BACKGROUND OF THE INVENTION

Embodiments of the present invention relate generally to immunology and in particular to the field of intravenous immunoglobulins.

The immune system has two branches: innate immunity that is able to act immediately upon exposure to foreign organisms, and adaptive or acquired immunity that requires previous exposure in order to act. The two arms work together to protect the host from external threats such as microbes, as well as internal threats such as damaged or altered tissues.

Circulating in the blood are proteins produced by cells of the adaptive and innate immune systems. These proteins work together to keep the body healthy and free from infection. The complement system is one of the first parts of innate immunity to interact with an invading microorganism. Immunoglobulins are plasma proteins that are produced in response to exposure to foreign substances and have high specificity for the antigens that stimulated their production. These immunoglobulins are the antibodies that form a major component of the adaptive immune system. Together, antibodies and complement bind to and either cause direct killing of a microbe, or interact with white blood cells to cause ingestion and killing by the cells.

Patients with primary immune deficiencies lack the ability to make the immunoglobulin antibodies that would protect them and thus have many infections. Intravenous immunoglobulin preparations (IVIg) are often used to treat patients presenting with primary immune deficiency disorders (PIDD), with the goal of replacing missing immune components. One form of PIDD is Common Variable Immune Deficiency (CVID), which is typically characterized by infections, gastrointestinal disorders, autoimmune diseases and increased susceptibility to malignancies.

Although IVIg treatments are currently available and provide real benefits to patients in need thereof, many advances may still be made to provide improved IVIg treatments, as well as improved processing and diagnostic techniques. Embodiments of the present invention provide solutions to at least some of these outstanding needs.

### BRIEF SUMMARY OF THE INVENTION

IVIg treatment provides a relatively safe form of therapy. Headaches and fatigue are the most common side effects, and the more severe forms of adverse events, such as aseptic meningitis, are rare. In a group of patients receiving IVIg treatment, an association was discovered between certain treatment side effects and complement protein activity or levels in the patient.

In one aspect, embodiments of the present invention encompass methods of providing an immunotherapy treatment to a patient. Exemplary methods include determining or obtaining a level of C1 esterase inhibitor or inhibitor activity in the patient, and determining the immunotherapy treatment for the patient based on the level of C1 esterase inhibitor or inhibitor activity. The immunotherapy treatment may include an intravenous immunoglobulin dosing protocol. The method may further include administering the immunotherapy treatment to the patient. In some cases, the immunotherapy treatment may also include a C1 esterase inhibitor dosing protocol. In some cases, the treated patient may have been diagnosed with a common variable immunodeficiency, and optionally, may have been selected for treatment based on the diagnosis. In some cases, the treated patient may have been diagnosed with an autoimmune disease, condition, or anomaly, and optionally, may have been selected for treatment based on the diagnosis. In some cases, the patient may have presented with a condition selected from the group consisting of arthralgia, fatigue, and malignancy.

In another aspect, embodiments of the present invention encompass methods for providing an immunotherapy treatment to a patient, which include determining or obtaining a level of complement protein or protein activity in the patient, and determining the immunotherapy treatment for the patient based on the level of complement protein or protein activity. In some cases, the immunotherapy treatment may include an intravenous immunoglobulin dosing protocol. Methods may also include administering the immunotherapy treatment to the patient.

In a further aspect, embodiments of the present invention encompass methods of providing an immunotherapy treatment to a patient, which include determining or obtaining a level of C1 esterase inhibitor or inhibitor activity in the patient after the patient has received an intravenous immunoglobulin treatment, and determining the immunotherapy treatment based on the determined level of C1 esterase inhibitor or inhibitor activity. The immunotherapy treatment may include a C1 esterase inhibitor dosing protocol. Methods may also include administering the immunotherapy treatment to the patient. In some cases, the immunotherapy treatment also includes an intravenous immunoglobulin dosing protocol. In some cases, the treated patient may have been diagnosed with a common variable immunodeficiency, and optionally, may have been selected for treatment based on the diagnosis. In some cases, the treated patient may have been diagnosed with an autoimmune disease, condition, or anomaly, and optionally, may have been selected for treatment based on the diagnosis. In some cases, the patient may have presented with a condition selected from the group consisting of arthralgia, fatigue, and malignancy.

In one aspect, embodiments of the present invention encompass methods of providing an immunotherapy treatment to a patient, which include determining or obtaining a level of complement protein or protein activity in the patient after the patient has received an intravenous immunoglobulin treatment, and determining the immunotherapy treatment based on the determined level of complement protein or protein activity. The immunotherapy treatment may include a C1 esterase inhibitor dosing protocol. The method may also include administering the immunotherapy treatment to the patient.

In still another aspect, embodiments of the present invention encompass methods of evaluating one or more production processes for an intravenous immunoglobulin preparation. Exemplary methods may include determining or obtaining a first level of C1 esterase inhibitor or inhibitor activity in a patient before the patient has received a treatment with the intravenous immunoglobulin preparation, determining or obtaining a second level of C1 esterase inhibitor or inhibitor activity in the patient after the patient has received the treatment with the intravenous immunoglobulin preparation, and evaluating the production process for the intravenous immunoglobulin preparation, based on a comparison between the first and second levels of C1 esterase inhibitor or inhibitor activity in the patient, or by comparing the first and second levels of C1 esterase inhibitor or inhibitor activity in the patient. Methods may also include providing or transmitting an alert if a difference between the first and second levels of C1 esterase inhibitor or inhibitor activity in the patient exceeds a predetermined threshold. In some instances, a difference between the first and second levels of C1 esterase inhibitor or inhibitor activity in the patient exceeding a predetermined threshold indicates a deficiency or anomaly in the production process.

In another aspect, embodiments of the present invention encompass methods of evaluating a transport process for an intravenous immunoglobulin preparation. An exemplary method may include obtaining or determining a first level of C1 esterase inhibitor or inhibitor activity in a patient before the patient has received a treatment with the intravenous immunoglobulin preparation, obtaining or determining a second level of C1 esterase inhibitor or inhibitor activity in the patient after the patient has received the treatment with the intravenous immunoglobulin preparation, and evaluating the transport process for the intravenous immunoglobulin preparation based on a comparison between the first and second levels of C1 esterase inhibitor or inhibitor activity in the patient, or by comparing the first and second levels of C1 esterase inhibitor or inhibitor activity in the patient.

In another aspect, embodiments of the present invention encompass methods of evaluating a storage process for an intravenous immunoglobulin preparation. Exemplary methods include obtaining or determining a first level of C1 esterase inhibitor or inhibitor activity in a patient before the patient has received a treatment with the intravenous immunoglobulin preparation, obtaining or determining a second level of C1 esterase inhibitor or inhibitor activity in the patient after the patient has received the treatment with the intravenous immunoglobulin preparation, and evaluating the storage process for the intravenous immunoglobulin preparation based on a comparison between the first and second levels of C1 esterase inhibitor or inhibitor activity in the patient, or by comparing the first and second levels of C1 esterase inhibitor or inhibitor activity in the patient.

In another aspect, embodiments of the present invention encompass methods of evaluating a handling process for an intravenous immunoglobulin preparation. Exemplary methods include obtaining or determining a first level of C1 esterase inhibitor or inhibitor activity in a patient before the patient has received a treatment with the intravenous immunoglobulin preparation, obtaining or determining a second level of C1 esterase inhibitor or inhibitor activity in the patient after the patient has received the treatment with the intravenous immunoglobulin preparation, and evaluating the handling process for the intravenous immunoglobulin preparation based on a comparison between the first and second levels of C1 esterase inhibitor or inhibitor activity in the patient, or by comparing the first and second levels of C1 esterase inhibitor or inhibitor activity in the patient.

In another aspect, embodiments of the present invention encompass methods of evaluating production processes for intravenous immunoglobulin preparations. Exemplary methods include obtaining or determining a preliminary measure of C1 esterase inhibitor or inhibitor activity in a first patient group before the first patient group has received a treatment with intravenous immunoglobulin provided by a first production process, obtaining or determining a preliminary measure of C1 esterase inhibitor or inhibitor activity in a second patient group before the second patient group has received a treatment with intravenous immunoglobulin provided by a first production process, obtaining or determining a subsequent measure of C1 esterase inhibitor or inhibitor activity in the first patient group after the first patient group has received treatment with the intravenous immunoglobulin provided by a first production process, where a difference between the first patient group preliminary and subsequent measures providing a first marker for the first production process, obtaining or determining a subsequent measure of C1 esterase inhibitor or inhibitor activity in the second patient group after the second patient group has received treatment with the intravenous immunoglobulin provided by a second production process, where a difference between the second patient group preliminary and subsequent measures providing a second marker for the second production process, and evaluating the first and second production processes based on a comparison between the first and second markers, or by comparing the first and second markers.

In another aspect, embodiments of the present invention encompass methods of evaluating a production process for an intravenous immunoglobulin preparation. Exemplary methods include obtaining or determining a preliminary measure of C1 esterase inhibitor or inhibitor activity in a patient group before the patient group has received a treatment with intravenous immunoglobulin provided by the production process, obtaining or determining a subsequent measure of C1 esterase inhibitor or inhibitor activity in the patient group after the patient group has received treatment with the intravenous immunoglobulin provided by the first production process, where a difference between the patient group preliminary and subsequent measures provides a marker for the production process, and evaluating the production process based on a comparison between the marker and the reference marker, or by comparing the marker with a reference marker.

In another aspect, embodiments of the present invention encompass methods of evaluating a patient receiving an immunotherapy treatment. Exemplary methods include administering an intravenous immunoglobulin dosing protocol to the patient, and determining or obtaining a level of C1 esterase inhibitor or inhibitor activity in the patient following administration of the intravenous immunoglobulin dosing protocol.

The terms “invention,” “the invention,” “this invention” and “the present invention” used in this patent are intended to refer broadly to all of the subject matter of this patent and the patent claims. Statements containing these terms should be understood not to limit the subject matter described herein or to limit the meaning or scope of the patent claims. Embodiments of the invention covered by this patent are defined by the claims, not this Summary. This Summary is a high-level overview of various aspects of the invention and introduces some of the concepts that are further described in the Detailed Description section. This Summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

The above described and many other features and attendant advantages of embodiments of the present invention will become apparent and further understood by reference to the following detailed description when considered in conjunction with the accompanying drawings.

### BRIEF DESCRIPTION OF THE DRAWINGS

Illustrative embodiments of the present invention are described in detail below with reference to the following drawing figure.

FIG. 1 depicts aspects of patient data pertaining to complement related activity, according to embodiments of the present invention.

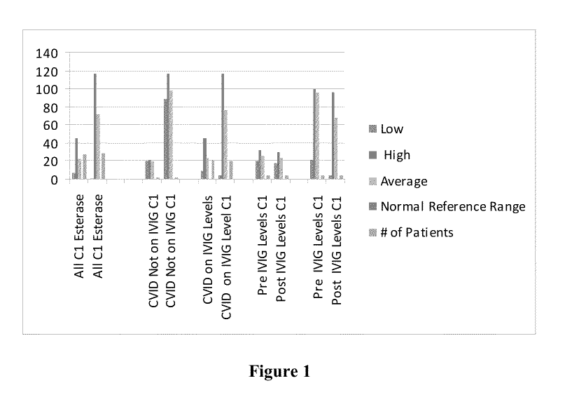

### DETAILED DESCRIPTION OF THE INVENTION

The precise mechanism of action of IVIg is complex and not yet fully understood. A number of mechanisms for the immune-modulatory action of IVIg have been reported, including Fc receptor blockade, enhancement of regulatory T-cells, inhibition of cytokines, accelerated clearance of autoantibodies, and modulation of adhesion molecules and cell receptors. Just as the exact mechanism of action of IVIg is not clear, the precise mechanism of action for side effects associated with IVIg is also not clear. At issue have been the common adverse events (AE's) related to infusion of IVIg.

IVIg remains relatively safe. A variety of common IVIg side effects have been reported, including headache, chills, myalgia, hives, tachycardia, and nausea. In some cases, more serious adverse events have been reported, including renal dysfunction, aseptic meningitis, and thrombotic events. Fortunately, these more severe forms of AE's remain rare. In some cases, adverse events can be attributed to infusion rates and product issues. In some cases, adverse events can be ameliorated by pre-medication. The underlying immunological mechanism of adverse events, however, has not been unraveled.

IVIg is commonly used for replacement therapy, for example in a dosing regimen that includes administration of IVIg at 400 to 800 mg/KgBW, and has been shown to reduce infection rates for all forms of PIDD. In the case of CVID, however, it has not been observed to be as effective in reducing the autoimmune component or the increased risk of malignancy for these patients.

IVIg is also used in higher doses, for example in a dosing regimen that includes administration of 1 to 2 g/KgBW, as an immune-modulator for other conditions, such as chronic inflammatory demyelinating polyneuropathy (CIDP) and autoimmune diseases. When an immune-modulatory dose is used to treat CVID for reduction in autoimmunity or prevention of malignancy, it has been observed to show little added benefit although it has been effective as a replacement therapy in reducing infection rates. Despite the use of IVIg for patients with CVID, autoimmunity and malignancy rates remain unchanged.

As used herein, the term “malignancy” or malignant refers to all types of cancer, neoplasm, or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas which can be treated with a combination of antineoplastic thiol-binding mitochondrial oxidant and an anticancer agent include a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with a combination of antineoplastic thiol-binding mitochondrial oxidant and an anticancer agent include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas which can be treated with a combination of antineoplastic thiol-binding mitochondrial oxidant and an anticancer agent include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniforni carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

As noted above, an association has been discovered between certain IVIg treatment side effects and complement protein activity or levels in the patient.

The complement system evolved very early in vertebrates and comprises an integral part of the innate immune system. Complement proteins form a trio of intersecting enzyme cascades that result in a number of protective mechanisms that bind to foreign surfaces and thus fight viruses, bacteria and other foreign substances. When the adaptive immune responses evolved, complement was able to interact with antibodies that had greater ability to distinguish specific targets, and one of the pathways of complement activation (Classical) became closely linked with the specific antibodies, “complementing” their action and adding their binding and killing strength to the defense.

The classical pathway proteins circulate throughout the body as inactive proenzymes. The first component of this pathway was named C1, and is made up of three different protein subunits that circulate together. C1q, the larger subunit, contains the recognition sites that bind to immunoglobulins when the latter are bound to their antigens. C1r and C1s, present in the C1 complex as two C1r-C1s dimers, are proenzymes that become active after the C1qrs complex binds. It is the subsequent action of C1s (C1-esterase) to start the classical pathway enzyme cascade by cleaving C4 and C2.

Complement activation is an integral part of the immune system's ability to fight viral and bacterial antigens (Ag). Antibody (Ab) production by activated T- and B-cells and subsequent binding is specific but it is the complement system that binds to the Ag/Ab complexes thereby completing the pathogenic destruction.

Inactive proteins of the complement system circulate throughout the body. In response to the Ag/Ab complex a protease, C1 esterase, cleaves the first protein, C1, to activate the complement cascade. This cleavage initiates the complement attack. There are over 30 complement proteins that can be activated and are specific for different forms of immune response but it is the C1 and C1 esterase interaction that triggers the complement cascade.

Once the complement cascade has been initiated there are certain proteins and other mechanisms that inhibit or slow down the attack at various places throughout the complement system. Without these checks in the system, uncontrolled complement activation can lead to inflammatory issues, autoimmune disorders, alterations of blood flow, or tissue destruction. These proteins beneficially slow down or stop the attack process, as uncontrolled complement activation can have very adverse effects on bodily systems.

C1 esterase inhibitor (C1-INH) is the major inhibitor of the classical pathway. In addition to blocking the activity of C1r and C1s, C1-INH has several non-complement target proteases, including factor XIIa and kallikrein of the kinin pathway, plasmin and MASPs 1 and 3 of the lectin pathway of complement.

CI-INH belongs to the family of serpins. Recent studies suggest some anti-inflammatory function for this N-terminal, possibly explaining the effects of C1-INH in diseases other than hereditary angiodema (HAE). Acquired deficiency of C1-INH can accompany activation of complement with consumption of C1 and hyper-activation of the classical pathway. This relationship has also been suggested with autoimmune (autoantibodies against the inhibitor) or lympho-proliferative diseases.

The mechanism for depletion is as follows: C1-INH binds to the enzyme it is inhibiting, and the resulting complex of ENZ-INH is cleared from the circulation. Likewise, an INH molecule with an antibody attached to it is an immune complex, capable of activating complement and exacerbating the clearance process.

Reduced Levels of C1-INH and Complement Activation

Embodiments of the present invention encompass techniques for evaluating or determining whether a patient may be at heightened risk for experiencing an adverse event associated with IVIg therapy, based on reduced levels of C1-INH and complement activation in the patient. For example, embodiments may include evaluating or determining whether a patient may be at heightened risk for experiencing headache, chills, myalgia, hives, tachycardia, nausea, renal dysfunction, aseptic meningitis, thrombotic events, stroke, or other inflammatory conditions, in association with receiving IVIg therapy. In view of evidence for an autoantibody mediated C1-INH deficiency, it may be possible to evaluate such risk based on autoantibody test results. Relatedly, because C1-INH has an effect on C1r and C1s activity, may it be possible to evaluate risk based on C1r/C1s activity, or other proteases which C1-INH inhibits, for example, factor XIIa. Embodiments of the present invention also encompass the use or evaluation of other elements associated with or implicated by the C1-INH regulatory pathway, such as C1-INH analogues and/or C1-INH targets or complement protein such as C1r/s.

Embodiments of the present invention also encompass techniques for determining whether a patient with CVID may be at heightened risk for experiencing arthralgia, fatigue, stroke, or malignancy in association with low levels of C1-INH and complement activation, for example based on the production of auto-antibodies to C1-INH contributing to the inflammatory pathways.

Embodiments of the present invention encompass methods for determining effects that certain production, storage, transport, or handling procedures may have on the content or activity of an intravenous immunoglobulin preparation. Relatedly, an exemplary biomarker for IVIg manufacturing production is based on the down-regulation, decrease, or deficiency of C1-INH in patients receiving IVIg treatment.

Without being bound by any particular theory, it is believed that C1-INH may be consumed by anti-idiotypic autoantibodies and it is these immune complexes that fix C1q and consume C1-INH. Embodiments of the present invention encompass techniques for altering or modulating the depletion of C1-INH.

Embodiments of the present invention further encompass treatments that combine the administration of IVIg with the administration of a C1 esterase inhibitor (e.g. Cinryze®) for those patients with low levels of C1-INH. Such treatments can be provided to reduce the likelihood of such patients experiencing severe adverse events, such as aseptic meningitis. Infusions of or administration protocols involving C1 esterase inhibitor combined with IVIg for CVID may reduce the autoimmune or malignancy risks that are currently not reduced by IVIg therapy alone. Embodiments also encompass IVIg and C1-INH combination therapy in patients with low levels of C1-INH, to reduce risk of other side effects, such as headache, chills, myalgia, hives, tachycardia, nausea, renal dysfunction, aseptic meningitis, thrombotic events, stroke, and other conditions associated with inflammation.

Embodiments of the present invention further encompass systems, devices, means, and methods for reducing or preventing mechanical agitation, shaking, or vibration in IVIg preparations during the production, storage, transport, or handling thereof.

Embodiments of the present invention further encompass systems, devices, means, and methods for reducing or preventing excess temperatures, or temperature variations or fluctuations, in IVIg preparations during the production, storage, transport, or handling thereof.

Embodiments of the present invention further encompass systems, devices, means, and methods for reducing or preventing immune complex formation, aggregates, and the like, during the production, storage, transport, or handling of intravenous immunoglobulin preparations. In some embodiments, the device may be a column, filter or other substrate on which aggregates (e.g. antibody aggregates, immune complexes) are immobilized and thereby separated from the intravenous immunoglobulin preparation. Where the device is a filter, any filter commonly known and used in the art to separate antibody aggregates from aqueous solutions may be used. In some embodiments, the filter has a pore size excluding antibody aggregates. In some embodiments, the filter has a pore size of about 0.2 um. In some embodiments, the intravenous immunoglobulin dosing protocol includes separating an intravenous immunoglobulin preparation from antibody aggregates, thereby forming a filtered intravenous immunoglobulin preparation and administering the filtered intravenous immunoglobulin preparation to a patient. Without being bound by any specific mechanism of action, it is believed that certain mechanical agitations such as shaking, and/or certain temperature conditions or variations, which may occur for example during production, processing, shipment, storage, or handling, can alter intravenous immunoglobulin products or components, leading to or amplifying aggregates, immune complexes, and the like, within the product itself, which may trigger the complement system, for example by causing, when infused in a patient, an increased usage of C1-INH in the immune cascade leading to side effects. Hence, immunoglobulin products or preparations can be shipped, stored, or otherwise contained in packaging that minimizes, reduces, or inhibits mechanical agitation or shaking For example, preparations can be packaged in containers with anti-shock or anti-vibration materials such as foams, pads, gels, and the like. Similarly, immunoglobulin products or preparations can be shipped, stored, or otherwise contained in packaging that minimizes, reduces, or inhibits temperature fluctuations or that helps to maintain the preparations at within a desired temperature range. Such packaging may include temperature control features such as cooling devices, heating devices, and/or insulating devices. In some cases, the packaging can be configured to maintain the preparations at a temperature of about 4 degrees Celsius. In some cases, the packaging can be configured to maintain the preparations at a temperature within a range from about 2 to about 4 degrees Celsius. In some cases, the packaging can be configured to maintain the preparations at a temperature within a range from about 2 to about 8 degrees Celsius. In some cases, the packaging can be configured to maintain the preparations at a temperature of less than about 25 degrees Celsius. In some cases, the packaging can be configured to protect the preparations from freezing.

Patient Data

Table 1 provides the results of studies involving patients having immune deficiencies or receiving intravenous immunoglobulin treatment. In each of these cases, the patients presented with autoimmune markers, such as antinuclear antibodies (ANA) or anti-DNA (anti-dsDNA) antibodies.

TABLE 1

| | | | | Normal | |
|---|---|---|---|---|---|
| | | | | Reference | # of |
| | Low | High | Average | Range | Patients |
| All C1 Esterase Function | 7 | 46 | 22.4 | 21-19 | 28 |
| All C1 Esterase Inhibitor | 1 | 117 | 71.93 | >67 | 29 |
| CVID Not on IVIG C1 | 20 | 22 | 21 | 21-19 | 3 |
| Esterase Function | | | | | |
| CVID Not on IVIG C1 | 89 | 117 | 99.3 | >67 | 3 |
| Esterase Inhibitor | | | | | |
| CVID on IVIG Levels C1 | 10 | 46 | 24 | 21-19 | 22 |
| EsteraseFunction | | | | | |
| CVID on IVIG Level C1 | 5 | 117 | 76.7 | >67 | 21 |
| Esterase Inhibitor | | | | | |
| Pre IVIG Levels C1 | 21 | 32 | 27 | 21-19 | 5 |
| Esterase Function | | | | | |
| Post IVIG Levels C1 | 18 | 30 | 23.8 | 21-19 | 5 |
| Esterase Function | | | | | |
| Pre IVIG Levels C1 | 22 | 100 | 96.2 | >67 | 5 |
| Esterase Inhibitor | | | | | |
| Post IVIG Levels C1 | 5 | 97 | 69.2 | >67 | 5 |
| Esterase Inhibitor | | | | | |

The values for C1 esterase function are provided in units of milligrams per deciliter (mg/dL). The values for C1 esterase inhibitor are provided in units of percent mean of normal values.

C1 esterase function and inhibitor levels can be determined by various immunochemical techniques. In some cases, the inhibitor function can be determined by chromogenic assay or enzyme-linked immunosorbent assay (ELISA or EIA). In some cases, the C1 esterase inhibitor levels can be determined by radial immunodiffusion (RID).

It was discovered that patients who have developed severe adverse events with IVIg, including aseptic meningitis, have low levels and function of C1-INH. For example, in four patients presenting with aseptic meningitis following treatment with IVIg, a first patient exhibited C1-INH levels ranging from 10-12 (where test normal range was 11-26) and C1-INH function measures of 64 (where test normal range was 83-108), 23.5 (where test normal range was 0.89 to 36.1), and 13.3 (where test normal range was 0.3 to 46.3). A second patient exhibited a C1-INH level of 29 (where test normal range was 21-39) and a C1-INH function measure of 22 (where test normal range was >67) prior to IVIg infusion. Following IVIg infusion, the second patient exhibited a C1-INH level of 22 (where test normal range was 21-39) and a C1-INH function measure of 5 (where test normal range was >67). A third patient exhibited a C1-INH level of 17 (where test normal range was 21-39) and a C1-INH function measure of 92 (where test normal range was >67). A fourth patient exhibited a C1-INH level of 32 (where test normal range was 21-39) and a C1-INH function measure of 92 (where test normal range was >67) prior to IVIg infusion. Following IVIg infusion, the fourth patient exhibited a C1-INH level of 30 (where test normal range was 21-39) and a C1-INH function measure of 62 (where test normal range was >67).

Further, it was observed that patients with CVID and autoimmune diseases presented with low levels of C1-INH. Patients treated with IVIg were also observed to have reduced levels of C1-INH post-infusion compared to pre-infusion levels. What is more, it was observed that different IVIg preparations had different effects on the decrease of C1-INH. Relatedly, it was observed that a cluster of aseptic meningitis cases were associated with various batches of the same IVIg product that were transported with the same shipment method of product to the clinic.

While exemplary embodiments have been described in some detail, by way of example and for clarity of understanding, those of skill in the art will recognize that a variety of modification, adaptations, and changes may be employed. Different arrangements of the components depicted in the drawings or described herein, as well as components and steps not shown or described are possible. Similarly, some features and subcombinations are useful and may be employed without reference to other features and subcombinations. Embodiments of the invention have been described for illustrative and not restrictive purposes, and alternative embodiments will become apparent to readers of this patent. Accordingly, the present invention is not limited to the embodiments described herein or depicted in the drawings, and various embodiments and modifications can be made without departing from the scope of the claims below. Hence, the scope of the present invention should be limited solely by the claims.

What is claimed is:

1. A method of providing an immunotherapy treatment to a patient, comprising:
determining a level of C1 esterase inhibitor or inhibitor activity in the patient;
determining the immunotherapy treatment for the patient based on the level of C1 esterase inhibitor or inhibitor activity, wherein the immunotherapy treatment comprises an intravenous immunoglobulin dosing protocol; and
administering the immunotherapy treatment to the patient.

2. The method according to claim 1, wherein the immunotherapy treatment further comprises a C1 esterase inhibitor dosing protocol.

3. The method according to claim 1, wherein the patient is diagnosed with a common variable immunodeficiency.

4. The method according to claim 1, wherein the patient is diagnosed with an autoimmune disease.

5. The method according to claim 1, wherein the patient presents with a condition selected from the group consisting of arthralgia, fatigue, and malignancy.

6. A method of providing an immunotherapy treatment to a patient, comprising:
determining a level of complement protein or protein activity in the patient;
determining the immunotherapy treatment for the patient based on the level of complement protein or protein activity, wherein the immunotherapy treatment comprises an intravenous immunoglobulin dosing protocol; and
administering the immunotherapy treatment to the patient.

7. A method of providing an immunotherapy treatment to a patient, comprising:
determining a level of C1 esterase inhibitor or inhibitor activity in the patient after the patient has received an intravenous immunoglobulin treatment;
determining the immunotherapy treatment based on the determined level of C1 esterase inhibitor or inhibitor activity, wherein the immunotherapy treatment comprises a C1 esterase inhibitor dosing protocol; and
administering the immunotherapy treatment to the patient.

8. The method according to claim 7, wherein the immunotherapy treatment further comprises an intravenous immunoglobulin dosing protocol.

9. The method according to claim 7, wherein the patient is diagnosed with a common variable immunodeficiency.

10. The method according to claim 7, wherein the patient is diagnosed with an autoimmune disease.

11. The method according to claim 7, wherein the patient presents with a condition selected from the group consisting of arthralgia, fatigue, and malignancy.

12. A method of providing an immunotherapy treatment to a patient, comprising:
determining a level of complement protein or protein activity in the patient after the patient has received an intravenous immunoglobulin treatment;
determining the immunotherapy treatment based on the determined level of complement protein or protein activity, wherein the immunotherapy treatment comprises a C1 esterase inhibitor dosing protocol; and
administering the immunotherapy treatment to the patient.

13. A method of evaluating a production process for an intravenous immunoglobulin preparation, comprising:
obtaining a first level of C1 esterase inhibitor or inhibitor activity in a patient before the patient has received a treatment with the intravenous immunoglobulin preparation;
obtaining a second level of C1 esterase inhibitor or inhibitor activity in the patient after the patient has received the treatment with the intravenous immunoglobulin preparation; and
evaluating the production process for the intravenous immunoglobulin preparation by comparing the first and second levels of C1 esterase inhibitor or inhibitor activity in the patient.

14. The method according to claim 13, further comprising providing an alert if a difference between the first and second levels of C1 esterase inhibitor or inhibitor activity in the patient exceeds a predetermined threshold.

15. The method according to claim 13, wherein a difference between the first and second levels of C1 esterase inhibitor or inhibitor activity in the patient exceeding a predetermined threshold indicates a deficiency in the production process.

16. A method of evaluating a transport process for an intravenous immunoglobulin preparation, comprising:
obtaining a first level of C1 esterase inhibitor or inhibitor activity in a patient before the patient has received a treatment with the intravenous immunoglobulin preparation;
obtaining a second level of C1 esterase inhibitor or inhibitor activity in the patient after the patient has received the treatment with the intravenous immunoglobulin preparation; and
evaluating the transport process for the intravenous immunoglobulin preparation by comparing the first and second levels of C1 esterase inhibitor or inhibitor activity in the patient.

17. A method of evaluating a storage process for an intravenous immunoglobulin preparation, comprising:
obtaining a first level of C1 esterase inhibitor or inhibitor activity in a patient before the patient has received a treatment with the intravenous immunoglobulin preparation;
obtaining a second level of C1 esterase inhibitor or inhibitor activity in the patient after the patient has received the treatment with the intravenous immunoglobulin preparation; and
evaluating the storage process for the intravenous immunoglobulin preparation by comparing the first and second levels of C1 esterase inhibitor or inhibitor activity in the patient.

18. A method of evaluating a handling process for an intravenous immunoglobulin preparation, comprising:
obtaining a first level of C1 esterase inhibitor or inhibitor activity in a patient before the patient has received a treatment with the intravenous immunoglobulin preparation;
obtaining a second level of C1 esterase inhibitor or inhibitor activity in the patient after the patient has received the treatment with the intravenous immunoglobulin preparation; and
evaluating the handling process for the intravenous immunoglobulin preparation by comparing the first and second levels of C1 esterase inhibitor or inhibitor activity in the patient.

19. A method of evaluating production processes for intravenous immunoglobulin preparations, comprising:
obtaining a preliminary measure of C1 esterase inhibitor or inhibitor activity in a first patient group before the first patient group has received a treatment with intravenous immunoglobulin provided by a first production process;
obtaining a preliminary measure of C1 esterase inhibitor or inhibitor activity in a second patient group before the second patient group has received a treatment with intravenous immunoglobulin provided by a first production process;
obtaining a subsequent measure of C1 esterase inhibitor or inhibitor activity in the first patient group after the first patient group has received treatment with the intravenous immunoglobulin provided by a first production process, a difference between the first patient group preliminary and subsequent measures providing a first marker for the first production process;
obtaining a subsequent measure of C1 esterase inhibitor or inhibitor activity in the second patient group after the second patient group has received treatment with the intravenous immunoglobulin provided by a second production process, a difference between the second patient group preliminary and subsequent measures providing a second marker for the second production process; and
evaluating the first and second production processes by comparing the first and second markers.

20. A method of evaluating a production process for an intravenous immunoglobulin preparation, comprising:
obtaining a preliminary measure of C1 esterase inhibitor or inhibitor activity in a patient group before the patient group has received a treatment with intravenous immunoglobulin provided by the production process;
obtaining a subsequent measure of C1 esterase inhibitor or inhibitor activity in the patient group after the patient group has received treatment with the intravenous immunoglobulin provided by the first production process, a difference between the patient group preliminary and subsequent measures providing a marker for the production process;
evaluating the production process by comparing the marker with a reference marker.

21. A method of evaluating a patient receiving an immunotherapy treatment, comprising:
administering an intravenous immunoglobulin dosing protocol; and
determining a level of C1 esterase inhibitor or inhibitor activity in the patient following administration of the intravenous immunoglobulin dosing protocol.
